(19) United States
(12) Patent Application Publication
(10) Pub. No.: US 2026/0133183 A1
(43) Pub. Date: May 14, 2026

(54) ELECTRICAL SIGNAL COMMUNICATION PLATFORM FOR MIMICKING CARDIOMYOCYTE CLUSTERS

(71) Applicant: Pengfei LU, Shihezi City (CN)

(72) Inventors: Pengfei LU, Shihezi City (CN); Ping XU, Shihezi City (CN); Fuqiang HU, Shihezi City (CN); Yongqiang ZHAO, Shihezi City (CN); Ziwei WANG, Shihezi City (CN); Hang ZHANG, Shihezi City (CN)

(73) Assignee: Pengfei LU, Shihezi City, XJ (CN)

(21) Appl. No.: 19/437,272

(22) Filed: Dec. 30, 2025

(30) Foreign Application Priority Data
Feb. 25, 2025 (CN) 202510208163.7

(51) Int. Cl. G01N 33/50; B01L 3/00

(52) U.S. Cl. CPC G01N 33/5091; B01L 3/508; B01L 2300/0645

(57) ABSTRACT

The present disclosure relates to the field of bioengineering technology, and particularly to an electrical signal communication platform for mimicking cardiomyocyte clusters. The transmitting end employs an Arduino Mega 2560™ main control board and an L298N motor drive module, the main control board sends a pulse signal to the motor drive module, and outputs a pulse waveform to a communication channel through the motor drive module; the communication channel employs a plurality of cell clusters, configured for receiving the pulse waveform output by the motor drive module and transmitting it to the receiving end; and the receiving end employs an oscilloscope to receive the pulse waveform passing through the communication channel, to demodulate the pulse waveform and send a demodulation result to a display device. The present disclosure provides multi-angle data acquisition to enhance the comprehensiveness of analysis, and facilitates a more accurate simulation of cardiomyocyte pacing and communication.

### TECHNICAL FIELD

The present disclosure relates to the field of bioengineering technology, and particularly to an electrical signal communication platform for mimicking cardiomyocyte clusters.

### BACKGROUND

A pacemaker is a medical device for treating arrhythmias by sending electrical stimulation signals to cardiomyocytes to maintain a normal heart rhythm. Conventional pacemakers primarily target myocardial tissue for stimulation. Nevertheless, as biomedical research has advanced, scientists have gradually acknowledged the significance of electrical signal communication among cardiomyocytes in cardiac function. In vivo, it is ubiquitous for cells to generate action potentials and communicate with one another. For instance, in the heart, an action potential generated by one cardiomyocyte is transmitted to multiple adjacent cells via gap junctions, enabling coordinated contractions of the heart. This mechanism is essential not only for normal cardiac pacing but also for advanced brain functions, including motivation, memory, and learning. By studying the cellular action potentials and the transmission of intercellular communication signals, it can be understood how complex cellular networks respond rapidly and effectively to external stimuli, thereby ensuring the adaptability and survival of organisms.

In recent years, research on cardiomyocyte communication has grown significantly. Nevertheless, there remains a lack of technical equipment capable of effectively mimicking and measuring intercellular electrical signal transmission. Existing studies predominantly rely on virtual simulations or single-cell-based simulation devices, which have significant limitations. For instance, currently available platforms mainly focus on the electrical stimulation of cells, such as using electrical signals to stimulate cardiomyocytes and make them pace, but there is no communication platform specifically for cardiomyocyte communication. Consequently, when studying the transmission mechanisms of electrical signals between cells and optimizing the action potential waveforms of cardiomyocyte clusters, the conventional single-cell simulation environments cannot accurately reproduce the complex cell communication environment.

### SUMMARY

In order to solve the problems existing in the prior art, the present disclosure provides an electrical signal communication platform for mimicking cardiomyocyte clusters. In the platform, a transmitting end employs an Arduino Mega 2560™ main control board and an L298N motor drive module, the main control board sends a pulse signal to the motor drive module, and outputs a pulse waveform to a channel through the motor drive module; a communication channel employs multiple cell clusters, configured for receiving the pulse waveform output by the motor drive module and transmitting it to a receiving end; the receiving end employs an oscilloscope to receive the pulse waveform passing through the communication channel, to demodulate the pulse waveform, and send a demodulation result to a display device. The present disclosure provides multi-angle data acquisition to enhance the comprehensiveness of analysis and facilitate a more accurate simulation of cardiomyocyte pacing and communication.

The present disclosure employs the following technical solution: an electrical signal communication platform for mimicking cardiomyocyte clusters, including: a transmitting end, a communication channel and a receiving end;
the transmitting end employs an Arduino Mega 2560™ main control board and an L298N motor drive module, the main control board sends a pulse signal to the motor drive module, and outputs a pulse waveform to a communication channel through the motor drive module;
the communication channel employs multiple cell clusters, configured for receiving the pulse waveform output by the motor drive module and transmitting it to the receiving end;
the receiving end employs an oscilloscope to receive the pulse waveform passing through the communication channel, to demodulate the pulse waveform and send a demodulation result to a display device.

Further, the transmitting end further includes: a waveform selective switch and a waveform control switch;
the waveform selective switch is connected to ports 4, 6 and 8 of the main control board, respectively, and configured for selecting a reference waveform type corresponding to the pulse signal sent by the main control board to the motor drive module;
the waveform control switch is connected to ports 5, 7 and 9 of the main control board, respectively, and configured for modifying an amplitude and a frequency of the reference waveform corresponding to the pulse signal sent by the main control board to the motor drive module.

Further, the transmitting end further includes: a waveform adjustment potentiometer;
the waveform adjustment potentiometer is connected to ports AD2, AD3, AD0, AD1, AD4 and AD5 of the main control board, respectively, and the main control board adjusts the amplitude and frequency of the reference waveform corresponding to the send pulse signal according to a value of the waveform adjustment potentiometer.

Further, ports IN1, IN2, IN3, IN4, ENA, and ENB of the motor drive module are connected to ports 12, 13, 10, 11, 2, and 3 of the main control board, respectively, enabling the main control board to send the pulse signal to the motor drive module.

Further, when the main control board sends the pulse signal to the motor drive module, further including:
constructing a filter switch circuit by employing a relay and a transistor, and inputting the pulse signal into a filter circuit for filtering according to the filter switch circuit; where the filter circuit is constructed using a multi-cascade RC low-pass filter.

Further, among multiple cell clusters in the communication channel, test points are arranged between every two cell clusters, and configured for connecting the oscilloscope to detect the pulse waveform in the communication channel.

The beneficial effects of the present disclosure are as follows: the simulation platform of the present disclosure employs a single/multi-cell model incorporating cardiomyocytes, enabling it to reflect the influence of the electrical signal on the action potential threshold of a single cell and the transmission of the electrical signal in the multicellular environment. Additionally, the platform of the present disclosure supports the adjustment of the amplitude of the electrical signal waveform to simulate various physiological conditions: when the amplitude is small, which does not generate an action potential in the cell, the electrical signal can be transmitted along the cell without producing pacing effect; when the amplitude is larger, the action potential can be generated to produce pacing effect on the cardiomyocytes. In summary, the present disclosure provides multi-angle data acquisition to enhance the comprehensiveness of analysis, facilitates a more accurate simulation of cardiomyocyte pacing and communication, and provides a novel direction for related research. Through the simulation platform of the present disclosure, researchers can more comprehensively explore the mechanisms of action potentials and communication in cardiomyocytes, providing a scientific basis for the treatment of heart diseases.

### BRIEF DESCRIPTION OF THE DRAWINGS

To explain the embodiments of the present disclosure or the technical solutions in the prior art more clearly, a brief introduction will be made to the accompanying drawings used in the embodiments or the description of the prior art. It is obvious that the drawings in the description below are only some embodiments of the present disclosure, and those ordinarily skilled in the art can obtain other drawings according to these drawings without creative work.

FIG. 1 is a schematic structural diagram of an electrical signal communication platform for mimicking cardiomyocyte clusters according to an embodiment of the present disclosure.

FIG. 2 is a schematic diagram of a filter circuit according to an embodiment of the present disclosure.

FIG. 3 is a schematic diagram of a switch circuit according to an embodiment of the present disclosure;

FIG. 4 is a circuit schematic diagram of a waveform selective switch and a waveform control switch according to an embodiment of the present disclosure;

FIG. 5 is a schematic diagram of a waveform adjustment circuit according to an embodiment of the present disclosure.

Reference numerals in figures: 1. a transmitting-end computer; 2. an Arduino Mega 2560™ main control board; 3. an L298N motor drive module; 4. a communication channel; 5. an oscilloscope; 6. a receiving-end computer.

### DETAILED DESCRIPTION OF THE EMBODIMENTS

In order to make the objectives, the technical solutions, and the advantages of the present disclosure clearer, the following clearly and completely describes the technical solutions in embodiments of the present disclosure with reference to the drawings of embodiments of the present disclosure. Apparently, the described embodiments are only some but not all of the embodiments of the present disclosure. All other embodiments obtained by those of ordinary skill in the art based on the embodiments of the present disclosure without involving any creative effort shall fall within the scope of protection of the present disclosure.

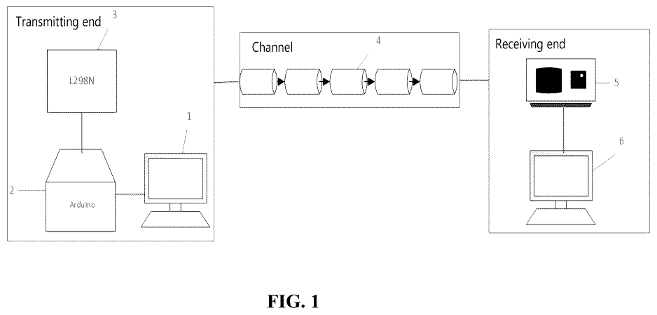

The flow diagram of the electrical signal communication platform for mimicking cardiomyocyte clusters of the embodiment of the present disclosure is shown in FIG. 1, including the transmitting end, the communication channel and the receiving end.

The transmitting end employs the Arduino Mega 2560™ main control board 2 and the L298N motor drive module 3, the Arduino Mega 2560™ main control board 2 sends a pulse signal to the L298N motor drive module 3, and outputs the pulse waveform to the communication channel 4 through the L298N motor drive module 3.

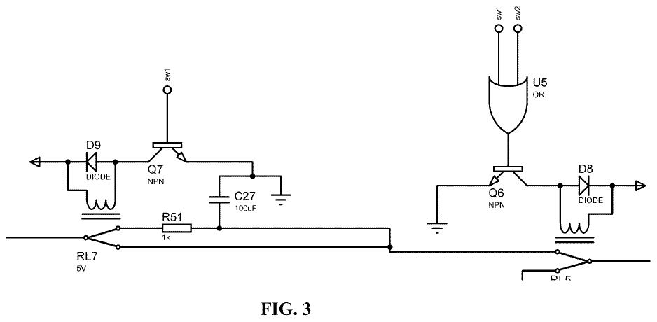

In the embodiment of the present disclosure, the L298N motor drive module 3 is employed to transmit electrical signals, and the Arduino Mega 2560™ main control board 2 load the Arduino program by connecting the transmitting-end computer 1 to achieve the modulation waveform and transmission function, and reads the waveform selection and the high and low levels of the control switch, and the value of the waveform adjustment potentiometer controls the waveform output from the L298N motor driver 3, specifically:
In the present disclosure, the ports IN1, IN2, IN3, IN4, ENA and ENB of L298N motor drive module 3 are connected to the ports 12, 13, 10, 11, 2 and 3 of Arduino Mega 2560™ main control board 2 respectively, so that the loaded Arduino program can output pulse width modulation (PWM) to L298N motor drive module 3. L298N motor drive module 3 can transmit three reference waveforms, including sine wave, square wave and triangular wave. Taking the sine wave as an example, the waveform to be filtered is output by the ports OUT1 and OUT2 of the L298N motor drive module 3 and passed through the filter circuit, the non-smooth waveform will become smoother. For some waveforms, such as square waves, the filtering will change the waveform characteristics of square waves and make them lose the characteristics of a fast rising-falling edge. In the application scenario of the present disclosure, the square wave may be used as a reference waveform, and its high-frequency harmonics will not cause significant interference to the communication channel or the receiving end, and no filtering is required. Therefore, the ports OUT3 and OUT4 of the L298N motor drive module 3 are used for outputting. In order to prevent the current from returning to the filter circuit, the switching circuit shown in FIG. 3 is also used for physical blocking in the embodiment of the present disclosure. Because the resistance and capacitance parameters of the triangular wave filter circuit are different from the parameters of the sine wave filter circuit, the ports OUT1 and OUT2 of the L298N motor drive module 3 cannot be directly used for output, so they are output by the ports OUT3 and OUT4. And the second set of filter circuit is designed, and the second set of switch circuit is used to physically block it from the square wave circuit. The specific circuit type can adjust the amplitude and frequency of the three preset waveforms according to the actual requirements to study the conduction characteristics of the cardiomyocytes electrical signal. The present disclosure is not limited to this.

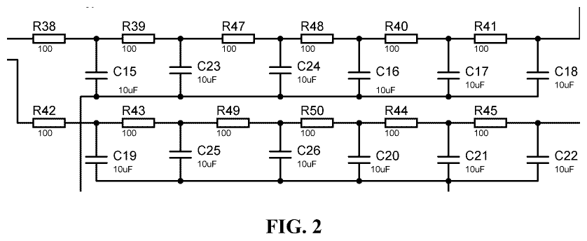

In the embodiment of the present disclosure, a filter circuit is shown in FIG. 2. The RC low-pass filter is used to smooth the high-frequency noise in the signal to ensure that the output signal is smoother and the waveform is distortion-free. The RC circuit is designed in series with resistors and capacitors. The specific parameter selection is determined according to the frequency requirements of the target waveform: multiple RC filter modules are designed to adapt waveforms in different frequency ranges. In order to improve the roll-off performance, the filtering effect can be improved by cascading multiple filters; the filtering module has a low cost and a high noise attenuation efficiency. The filter switch circuit composed of a relay and a triode is employed: it supports dynamic selection of whether to pass through the filter circuit, thus ensuring that the waveform without filtering is not affected by the filter circuit. The relay is a mechanical switch with electrical isolation advantages, which can safely switch high and low power loads. The triode provides the driving current required by the relay to ensure that the circuit switching is sensitive and reliable, while protecting the control circuit.

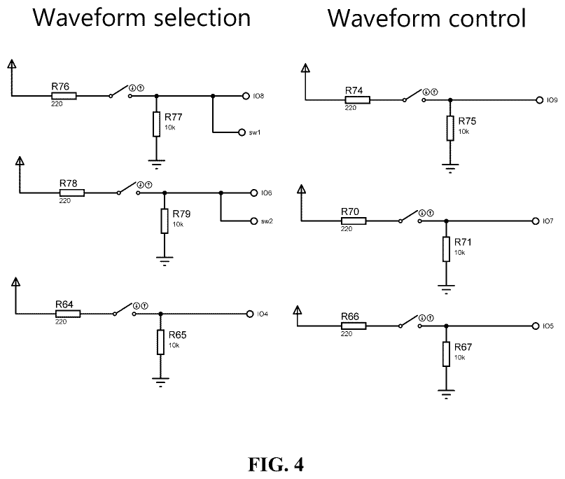

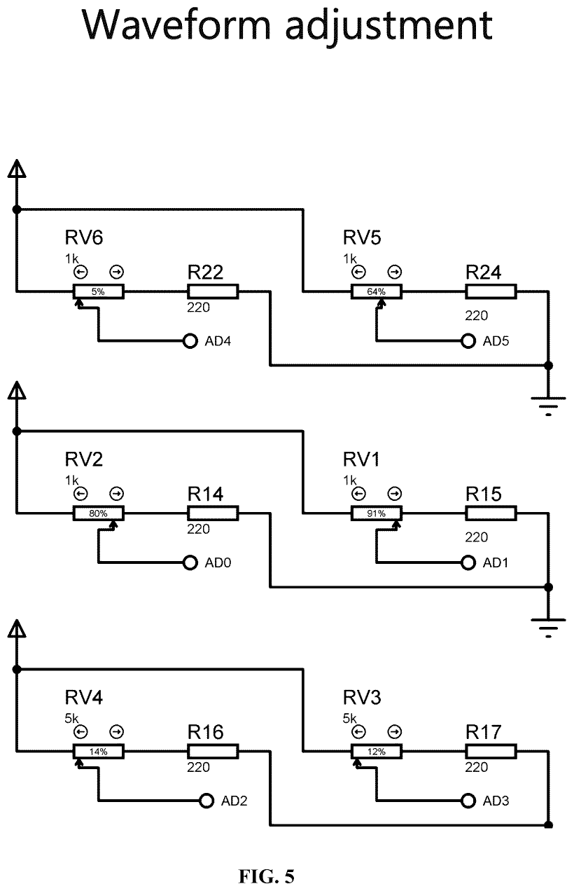

In the embodiment of the present disclosure, when the transmitting end outputs the pulse waveform, it also has the modulation and coding function of the pulse waveform. Firstly, the type of pulse waveform and whether it is modulated are controlled by waveform selective circuit and waveform control circuit, as shown in FIG. 4. Specifically: the waveform selective circuit controls which of the three reference waveforms is transmitted from the Arduino program to the L298N motor drive module 3. Each switch in the circuit controls one waveform, and is connected to the ports 4, 6, and 8 of the Arduino Mega 2560™ main control board 2, respectively. The waveform control circuit can control whether the current system allows the amplitude and frequency of the three reference waveforms to be modified. Each switch in the circuit controls one waveform, and is connected to the ports 5, 7, and 9 of the Arduino Mega 2560™ main control board 2, respectively. The embodiment of the present disclosure further modulates the frequency and amplitude of the output pulse waveform by setting the waveform adjustment circuit. A waveform adjustment circuit of the embodiment of the present disclosure is shown in FIG. 5. Arduino Mega 2560™ main control board 2 modifies the frequency and amplitude of the reference waveform by reading the value of the potentiometer in the circuit. Each potentiometer corresponds to three reference waveforms. According to the output pulse waveform type, the corresponding potentiometer is selected for reading, and is connected to the ports AD2, AD3, AD0, AD1, AD4 and AD5 of the Arduino Mega 2560™ main control board 2, respectively. Arduino Mega 2560™ main control board 2 will select and control the level of the switch and adjust the value of the potentiometer according to the reading waveform according to the reading waveform to transmit one of the three reference waveforms to the L298N motor drive module 3. The combination of the switch circuit and the filter circuit ensures that during signal transmission, waveforms requiring filtering can be processed by their dedicated filtering circuit. It ensures that waveforms remain undistorted, guaranteeing efficient signal transmission and real-time data processing.

The communication channel 4 employs multiple cell clusters, configured for receiving the pulse waveform output by the L298N motor drive module 3 and transmitting it to the receiving end.

In another embodiment of the present disclosure, the communication channel can also employ a 10 kΩ resistor to replace a cardiomyocyte cluster to simulate the cardiomyocyte scenario of the human heart, thereby restoring the real impedance scenario of the human heart cardiomyocytes transmitting electrical signals.

In the embodiment of the present disclosure, the communication channel arranges a test point every two cell clusters or resistors, so that the oscilloscope 5 at the receiving end can test the waveform signal through the test point.

The receiving end employs the oscilloscope 5 to receive the pulse waveform passing through the communication channel 4, to demodulate the pulse waveform and send the demodulation result to the display device.

In the embodiment of the present disclosure, the receiving end is responsible for receiving the electrical signal and demodulating the original information, and ensuring the signal sampling accuracy through the direct-connected load, which provides a reliable basis for subsequent waveform analysis and algorithm adjustment. The oscilloscope 5 is employed to detect the electrical signal passed through the communication channel 4. These signals can be connected to the receiving-end computer 6 to read out the data. After program processing and data statistics, the final result will be displayed on the display device. The optional display device includes but is not limited to the computer screen, mobile terminal or external display. Generally, in order to facilitate operation and viewing, the computer screen configured by the receiving-end computer 6 can be directly selected.

In view of the foregoing, a cardiomyocyte communication test platform of the present disclosure can be applied to study the electrical signal transmission in the cardiomyocyte environment, including determining which intensities and waveforms are capable of pacing cardiomyocytes; to study the pacing energy optimization of the pacemaker, and provide a verification device for the study of cardiomyocyte communication. Furthermore, by replacing cardiomyocytes with other human tissue cells, the platform can be further utilized to investigate the electrophysiological characteristics of other cell types.

The above examples are merely preferred examples of the present disclosure, but not intended to limit the present disclosure, and any modifications, equivalent replacements, improvements, etc. made within the spirit and principles of the disclosure should fall within the scope of protection of the present disclosure.

What is claimed is:

1. An electrical signal communication platform for mimicking cardiomyocyte clusters, comprising a transmitting end, a communication channel and a receiving end;
wherein the transmitting end employs an Arduino Mega 2560™ main control board and an L298N motor drive module, wherein the main control board sends a pulse signal to the motor drive module, and outputs a pulse waveform to a communication channel through the motor drive module;
wherein the communication channel employs a plurality of cell clusters, configured for receiving the pulse waveform output by the motor drive module and transmitting it to the receiving end;
wherein the receiving end employs an oscilloscope to receive the pulse waveform passing through the communication channel, to demodulate the pulse waveform and to send a demodulation result to a display device.

2. The electrical signal communication platform for mimicking cardiomyocyte clusters according to claim 1, wherein the transmitting end further comprises: a waveform selective switch and a waveform control switch;
wherein the waveform selective switch is connected to ports 4, 6 and 8 of the main control board, respectively, and is configured for selecting a reference waveform type corresponding to the pulse signal sent by the main control board to the motor drive module;
wherein the waveform control switch is connected to ports 5, 7 and 9 of the main control board, respectively, and configured for modifying an amplitude and a frequency of the reference waveform corresponding to the pulse signal sent by the main control board to the motor drive module.

3. The electrical signal communication platform for mimicking cardiomyocyte clusters according to claim 1, wherein the transmitting end further comprises: a waveform adjustment potentiometer;
wherein the waveform adjustment potentiometer is connected to ports AD2, AD3, AD0, AD1, AD4 and AD5 of the main control board, respectively, and the main control board adjusts an amplitude and a frequency of the reference waveform corresponding to the send pulse signal according to a value of the waveform adjustment potentiometer.

4. The electrical signal communication platform for mimicking cardiomyocyte clusters according to claim 1, wherein ports IN1, IN2, IN3, IN4, ENA, and ENB of the motor drive module are connected to ports 12, 13, 10, 11, 2, and 3 of the main control board, respectively, enabling the main control board to send the pulse signal to the motor drive module.

5. The electrical signal communication platform for mimicking cardiomyocyte clusters according to claim 1, wherein when the main control board sends the pulse signal to the motor drive module, further comprising:
constructing a filter switch circuit by employing a relay and a transistor, and inputting the pulse signal into a filter circuit for filtering according to the filter switch circuit; wherein the filter circuit is constructed using a multi-cascade RC low-pass filter.

6. The electrical signal communication platform for mimicking cardiomyocyte clusters according to claim 1, wherein among a plurality of cell clusters in the communication channel, test points are arranged between every two cell clusters, and configured for connecting the oscilloscope to detect the pulse waveform in the communication channel.
